(19) United States
(12) Patent Application Publication
(10) Pub. No.: US 2012/0315641 A1
(43) Pub. Date: Dec. 13, 2012

(54) Protein Markers for Lung Cancer Detection and Methods of Using Thereof

(75) Inventors: Steven M. Dubinett, Los Angeles, CA (US); Brian K. Gardner, Los Angeles, CA (US); David Elashoff, Los Angeles, CA (US); Kostyantyn Krysan, Los Angeles, CA (US)

(73) Assignee: The Regents of the University of California

(21) Appl. No.: 13/520,660

(22) PCT Filed: Jan. 7, 2011
(86) PCT No.: PCT/US2011/020463

§ 371 Date: Aug. 28, 2012

Related U.S. Application Data
(60) Provisional application No. 61/293,550, filed on Jan. 8, 2010.

(51) Int. Cl. G01N 33/574; G06F 19/10; C12Q 1/68

(52) U.S. Cl. 435/6.12; 436/501; 702/19

(57) ABSTRACT

Disclosed herein are methods, devices and kits for detecting, diagnosing, or categorizing a subject as having lung cancer. As disclosed herein, at least three of the following protein biomarkers: VEGF, CGSF, MIG, RANTES, IL-2, IL-3 and MDC, are used to determine whether a subject at high-risk for lung cancer likely has lung cancer, such as stage I non-small cell lung cancer.

### CROSS-REFERENCE TO RELATED APPLICATIONS

This application claims the benefit of U.S. Application Ser. No. 61/293,550, filed 8 Jan. 2010, which is herein incorporated by reference in its entirety.

### ACKNOWLEDGEMENT OF GOVERNMENT SUPPORT

This invention was made with Government support under Grant Nos. CA 090338 and DA 016339, awarded by the National Institutes of Health. The Government has certain rights in this invention.

This work was also supported by the U.S. Department of Veterans Affairs, and the Federal Government has certain rights of this invention.

### BACKGROUND OF THE INVENTION

1. Field of the Invention

The present invention generally relates to protein markers and methods for the detection of lung cancer.

2. Description of the Related Art

Lung cancer is the leading cause of death from cancer in the United States. Currently, the overall five-year survival rate is only 14%, and this figure has not changed significantly over the last three decades. At time of clinical presentation, only about 25% of subjects have surgically resectable lung cancer. See Birring, et al. (2005) Thorax. 60(4):268-269. Moreover, subjects having pathologic stage IA lung cancers who undergo surgical resection only have a five-year survival rate of 67%. It is estimated that it can take up to 8 years for a lung carcinoma to reach clinical detection providing an opportunity for early detection.

US 20090068685 discloses various biomarkers which are differentially expressed among lung cancer subjects vs. asthma subjects and lung cancer subjects vs. normal subjects. Unfortunately, US 20090068685 does not disclose anything about any differential expression patterns between lung cancer subjects vs. subjects at high risk for lung cancer (who may or may not have indeterminate pulmonary nodules). As such, the biomarker panels disclosed in US 20090068685 cannot be used to accurately determine whether a subject at high risk for lung cancer actually has lung cancer. This is because different factors, such as smoking, cause one to have different biomarker expression profiles. The differential expression profile of one set of factors (e.g. asthma) can not be correlated to or suggest a differential expression profile of a different set of factors (exposure to cigarette smoke). In addition, the differential expression patterns of US 20090068685 cannot account for any similarities of biomarker expression patterns between high risk subjects and lung cancer subjects. Specifically, smoking causes chronic inflammation, deregulated cells, aberrant repair, increased product of cytokines and growth factors which are associated with the development of lung cancer. See Walser et al.(2008) Proc Am Thorac Soc 5(8):811-5; Auerbach et al. (1961) N Engl J Med 265:253-67; and Wistuba, II, (2007) Curr Mol Med 7(1):3-14. As such, it is unknown whether such biochemical and physiological effects will result in biomarker expression profiles which are indistinguishable between high risk subjects and subjects who have lung cancer.

Thus, a need exists for diagnostics and methods for the early detection of lung cancer in high risk subjects, including the detection of subclinical lung cancer.

### SUMMARY OF THE INVENTION

The present invention provides methods of detecting, diagnosing, or categorizing a subject as having a lung cancer which comprises determining the amounts of at least three of the following protein biomarkers: VEGF, CGSF, MIG, RANTES, IL-2, IL-3 and MDC, in a blood, serum or plasma sample from the subject, and determining whether the amounts are indicative of the lung cancer. In some embodiments, logistic regression analysis is used to calculate a predicted probability of the lung cancer. In some embodiments, the lung cancer is non-small cell lung cancer. In some embodiments, the amounts of VEGF, GCSF, MIG and RANTES are determined and logistic regression analysis is used to calculate a predicted probability of the lung cancer. In some embodiments, the lung cancer is stage I non-small cell lung cancer. In some embodiments, the amounts of IL-2, IL-3 and MDC are determined and logistic regression analysis is used to calculate a predicted probability of the lung cancer. In some embodiments, the subject is categorized as being at high risk for lung cancer. In some embodiments, the subject smokes or has smoked at least 20 packs of cigarettes, preferably at least 30 packs of cigarettes per year and is at least 35 years of age, preferably at least 45 years of age. In some embodiments, the amounts are indicative of the lung cancer where the predicted probability is greater than or equal to 0.6, preferably greater than or equal to 0.7, more preferably greater than or equal to 0.8, most preferably greater than or equal to 0.9. In some embodiments, the amounts are not indicative of the lung cancer where the predicted probability is less than or equal to 0.4, preferably less than or equal to 0.3, more preferably less than or equal to 0.2, most preferably less than or equal to 0.1.

In some embodiments, the methods further comprise determining the amounts of one or more of the following protein biomarkers: CXCL1 (GROα), CXCL3 (GROγ), CXCL5 (ENA-78), CCL1 (1309), CXCL11 (I-TAC), CXCL12 (SDF-1), CCL3 (MIP-1α), CCL4 (MIP-1β), CCL11 (eotaxin), CCL15 (MIP1δ), CCL19 (MIP3β), IL-4, IL-6, IL-7, IL-10, IL-12B (p40), IL-12 (p70), IL-13, IL-15, IL-17, GM-CSF, INF-γ, IL-1α, IL-1β, IL1Ra, and TNFβ, and determining whether the amounts are indicative of the lung cancer. In some embodiments, the methods further comprise determining the amounts of one or more of the following protein biomarkers: CXCL3 (GROγ), CCL3 (MIP-1α), CCL15 (MIP1δ), IL-6, IL-1α, and IL-1β, and determining whether the amounts are indicative of the lung cancer. In some embodiments, the methods further comprise determining the amounts of one or more miRNAs selected from the group consisting of miR-21, miR-25, miR-34a, miR-200c and miR-146b, and determining whether the amounts are indicative of the lung cancer.

In some embodiments, the present invention provides methods of monitoring or treating a subject who is at high risk of having a lung cancer, who has the lung cancer or who has had the lung cancer, which comprises determining the amounts of at least three of the following protein biomarkers: VEGF, CGSF, MIG, RANTES, IL-2, IL-3 and MDC, in a blood, serum or plasma sample from the subject, and treating the subject in accordance with the amounts.

In some embodiments, the present invention provides devices which comprise at least three capture reagents immobilized on one or more substrates, which each capture reagent specifically binds one protein biomarker selected from the group consisting of: VEGF, CGSF, MIG, RANTES, IL-2, IL-3 and MDC.

In some embodiments, the present invention provides kits which comprise reagents for assaying the amounts of at least three of the protein biomarkers as disclosed herein, e.g. at least three of the following protein biomarkers: VEGF, CGSF, MIG, RANTES, IL-2, IL-3 and MDC, packaged together.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 is a ROC curve for a predictive profile of stages I-IV NSCLC vs. control (non-NSCLC) using 33 biomarkers. This model provides a sensitivity of 87%, a specificity of 78% and an AUC of 0.92.

FIG. 2 is a ROC curve for a predictive profile model of stages I-IV NSCLC vs. control (non-NSCLC) using 4 biomarkers, i.e. VEGF, GCSF, MIG and RANTES. This model provides a sensitivity of 88%, a specificity of 79% and an AUC of 0.89.

FIG. 3 is a ROC curve for a predictive profile model of stage I NSCLC vs. control (non-NSCLC) using 3 biomarkers, i.e. IL-2, IL-3 and MDC. This model provides a sensitivity of 97%, a specificity of 77%, and an AUC of 0.93.

### DETAILED DESCRIPTION OF THE INVENTION

The present invention provides a plurality of protein biomarkers which may be used in diagnostic methods and devices for detecting and/or diagnosing whether a subject has non-small cell lung cancer (NSCLC). In particular, the expression levels of some or all of the biomarkers in a peripheral blood sample of a subject may be used to detect and/or diagnose whether the subject has NSCLC. Thus, the present invention also provides methods and devices for detecting and/or diagnosing whether a subject has NSCLC. As disclosed herein, the methods and devices of the present invention may be used to detect and/or diagnose whether a subject has stage I NSCLC.

Blood samples were collected from 89 human subjects who were clinically diagnosed as having lung cancer (lung cancer subjects) and 56 human subjects at high-risk for obtaining lung cancer (high-risk control subjects). Of the 89 lung cancer subjects, 31 subjects had stage I NSCLC. The high-risk control subjects were former smokers (at least a year of cessation) ages 45 years or older who smoked >30 packs of cigarettes per year prior to cessation. All control subjects underwent extensive screening to rule out pre-existing lung cancer, which was comprised of comprehensive clinical laboratory studies (complete blood count, chemistry panel, and coagulation studies), spirometry, helical CT scans and LIFE (fluorescence) bronchoscopy with BAL and bronchial biopsies.

All specimens utilized herein were collected from subjects who provided informed consent utilizing forms approved by the UCLA IRB. All specimens were complemented with collection of general health and medical information, including clinical and pathologic stages, medication history and comorbidity. The control specimens were comprised of former smokers at risk for lung cancer (≧30 pack years, age ≧45, smoking cessation of at least 1 year). All control subjects underwent extensive screening to rule out preexisting lung cancer which was included comprehensive clinical laboratory studies (complete blood count, chemistry panel, and coagulation studies), spirometry, helical CT scans and LIFE (fluorescence) bronchoscopy with BAL and bronchial biopsies. All lung cancer and control blood samples were collected and processed utilizing a standardized collection and storage protocol that was based on the blood sample collection protocol utilized by the NIH/NHLBI sponsored Lung Health Study trial (LHS). This protocol is designed to standardize collection methods to minimize sample degradation and sample variability due to non-standardized sample processing. All blood utilized herein was collected into BD Vacutainer® blood collection tubes (BD Diagnostics, Franklin Lakes, N.J.). The order of collection was red top first for serum collection followed by purple top for plasma. The red top serum collection tubes were allowed to sit at room temperature for 30 minutes to allow the blood to clot. The purple top tubes were centrifuged at 2,000 g for 10 minutes and the supernatant was collected. After incubation for clotting, the red top tubes were centrifuged at 2,000 g for 10 minutes and the supernatant was collected. To insure sample integrity all samples were processed and the serum and plasma were aliquoted into 1.0, 0.5 and 0.1 milliliter aliquots, frozen and stored at −80° C. within 2 hours of collection.

40 candidate protein biomarkers that could be associated with lung cancer progression or whose levels may be altered as a result of tumorigenesis were selected. The 40 candidate protein biomarkers are set forth in Table 1 as follows:

TABLE 1

| Name | Complete name and reference citation |
|---|---|
| CXCL1 (GROα)* | Chemokine (C—X—C motif) ligand 1, Haskill et al. (1990) |
| | PNAS USA 87 (19): 7732-6. |
| CXCL3 (GROγ)** | Chemokine (C—X—C motif) ligand 3, Smith et al. (2005) Am. |
| | J. Physiol. Heart and Circulatory Physiol. 289 (5): H1976-84. |
| CXCL5 (ENA-78)* | C—X—C motif chemokine 5, Chang et al. (1994) J. Biol. |
| | Chem. 269 (41): 25277-82. |
| CXCL8 (IL-8) | C—X—C motif chemokine 8, Modi et al. (1990) Hum. Genet. |
| | 84 (2): 185-7. |
| CCL1 (I309)* | Chemokine (C-C motif) ligand 1, Miller et al. (1992) PNAS |
| | USA 89 (7): 2950-4. |
| CCL2 (MCP-1) | Chemokine (C-C motif) ligand 2, Yoshimura et al. (1989) |
| | FEBS Lett. 244 (2): 487-93. |
| CXCL9 (MIG)* | Chemokine (C—X—C motif) ligand 9, Farber JM (1993) |
| | Biochem. Biophys. Res. Commun. 192 (1): 223-30. |
| CXCL10 (IP10) | C—X—C motif chemokine 10, Luster et al. (1985) Nature 315 |
| | (6021): 672-6. |
| CXCL11 (I-TAC)* | Chemokine (C—X—C motif) ligand 11, Cole et al. (1998) J. Exp. |
| | Med. 187 (12): 2009-21. |
| CXCL12 (SDF-1)* | Chemokine (C—X—C motif) ligand 12, Bleul et al. (1996) J. |
| | Exp. Med. 184 (3): 1101-9. |
| CCL3 (MIP-1α)** | Chemokine (C-C motif) ligand 3, Guan et al. (2001) J. Biol. |
| | Chem. 276 (15): 12404-9. |
| CCL4 (MIP-1β)* | Chemokine (C-C motif) ligand 4, Guan et al. (2001) J. Biol. |
| | Chem. 276 (15): 12404-9. |
| CCL5 (RANTES)* | Chemokine (C-C motif) ligand 5, Schall et al. (1988) J. |
| | Immunol. 141 (3): 1018-25. |
| CCL11 (eotaxin)* | Chemokine (C-C motif) ligand 11, Ponath et al. (1996) J. |
| | Clin. Invest. 97 (3): 604-12. |
| CCL15 (MIP1δ)** | Chemokine (C-C motif) ligand 15, Pardigol et al. (1998) |
| | PNAS USA 95: 6308-6313. |
| CCL19 (MIP3β)* | C-C motif chemokine 19, Yoshida et al. (1997) J. Biol. |
| | Chem. 272 (21): 13803-9. |
| CCL21 (6Ckine) | Chemokine (C-C motif) ligand 21, Hedrick et al. (1997) J. |
| | Immunol. 159 (4): 1589-93. |
| CCL22 (MDC)* | C-C motif chemokine 22, Godiska et al. (1997) J. Exp. |
| | Med. 185 (9): 1595-604. |
| IL-2* | Interleukin 2, Smith et al. (1983) J. Immunol. 131 (4): |
| | 1808. |
| IL-3* | Interleukin 3, Yang et al. (1986) Cell 47 (1): 3-10. |
| IL-4* | Interleukin 4, Howard et al. (1982) Lymphokine Res. 1 (1): |
| | 1-4. |
| IL-5 | Interleukin 5, Milburn et al. (1993) Nature 363 (6425): 172-176. |
| IL-6** | Interleukin 6, Ferguson-Smith et al. (1988) Genomics 2 (3): |
| | 203-8. |
| IL-7* | Interleukin 7, Goodwin et al. (1989) PNAS USA 86 (I): 302-6. |
| IL-10* | Interleukin 10, Pestka et al. (2004) Annu. Rev. Immunol. |
| | 22: 929-79. |
| IL-12B (p40)* | Subunit beta of interleukin 12, Entrez Gene: IL12B |
| | interleukin 12B (natural killer cell stimulatory factor 2, |
| | cytotoxic lymphocyte maturation factor 2, p40) |
| IL-12 (p70)* | Interleukin 12, Kalinski et al. (1997) J. Immunol. 159 (1): |
| | 28-35. |
| IL-13* | Interleukin 13, Minty et al. (1993) Nature 362 (6417): 248-50. |
| IL-15* | Interleukin 15, Grabstein et al. (1994) Science 264 (5161): |
| | 965-8. |
| IL-17* | Interleukin 17, Yao et al. (1996) J. Immunol. 155 (12): |
| | 5483-6. |
| bFGF | Basic fibroblast growth factor, Kurokawa et al. (1987) |
| | FEBS Lett. 213 (1): 189-94. |
| GCSF* | Granulocyte colony-stimulating factor, Nagata et al. (1986) |
| | Nature 319 (6052): 415-8. |
| GM-CSF** | Granulocyte-macrophage colony-stimulating factor, |
| | Esnault et al. (2002) Arch. Immunol. Ther. Exp. (Warsz.) |
| | 50 (2): 121-30. |
| INF-γ* | Interferon gamma, Ealick et al. (1991) Science 252 (5006): |
| | 698-702. |
| IL-1α** | Interleukin 1 alpha, March et al. (1985) Nature (6021): 641-7. |
| IL-1β** | Interleukin 1 beta, March et al. (1985) Nature (6021): 641-7. |
| IL1Ra* | Interleukin 1 receptor antagonist (1990) Nature 344, 6333-638 |
| TNFα | Tumor necrosis factor alpha, Pennica et al. (1984) Nature |
| | 312 (5996): 724-9. |
| TNFβ* | Tumor necrosis factor beta, Pennica et al. (1984) Nature |
| | 312 (5996): 724-9. |
| VEGF** | Vascular endothelial growth factor, Holmes et al. (2007) |
| | Cell Signal. 19 (10): 2003-2012. |

Stage I-IV NSCLC compared to control
*P < 0.05,
**P < 0.001
The sequences of each of the above-referenced proteins are herein incorporated by reference in their entirety.

Since these protein biomarker candidates are not specific cancer markers and whose levels can be altered in conditions and disorders other than lung cancer, use of one or more of these 40 candidate biomarkers in a biomarker panel might not reliably allow the detection or diagnosis of lung cancer in a subject with sufficient specificity and sensitivity. Thus, in order to determine whether one or more of these candidate biomarkers have any utility in detecting or diagnosing lung cancer, the following experiments were conducted.

To determine the concentration of these potential biomarkers in blood samples, a bead-based multiplexed immunoassay was used. Specifically, a LUMINEX immunoassay system was used to determine the concentration of each of the 40 biomarkers in serum samples obtained from lung cancer patients and individuals at elevated risk for lung cancer based on their smoking history and age.

Briefly, 100 μl of 1% bovine serum albumin/phosphate buffered saline (BSA/PBS) was added to the 96-well filter plate and removed by vacuum filtration. Then the bead set for the assay was added, typically 3,000 beads per analyte per well. The buffer the beads were suspended in was removed by vacuum filtration, and the beads were washed twice with 100 μl BSA/PBS before sample addition. Sample and standards (50 μl per well) were then added to the wells of the filter plate and incubated for 2 hr on a shaker at room temperature. A detection antibody cocktail solution was made by mixing together biotinylated antibodies for each of the target analytes in the assay. Following the first incubation the beads were washed 3 times with 100 μl BSA/PBS and then 25 μl of detection antibody cocktail was added for 2 hours. The beads were then washed 3 times with 100 μl BSA/PBS and incubated with 50 μl of streptavidin-R-phycoerythrin reporter (4 μg/ml in BSA/PBS) for 30 minutes. The plate was then washed with 100 μl BSA/PBS three times and the beads were resuspended in 125 μl of BSA/PBS for reading in the LUMINEX analyzer. Biomarker concentration values were then determined by an 8 point standard calibration curve using methods known in the art. In order to prevent experimental artifacts from corrupting the data, all sample groups (control and cancer) were randomized across the assay plates. In addition, all samples were run in triplicate, and these replicates were also randomized across the assay plates. Thus, sample groups were not processed separately, but samples and controls were instead processed together, so they were all treated in the same manner. This prevents processing errors from affecting specific groups of samples. In order to minimize the effects of assay variability, reference standards on each assay plate may be included so results can be normalized from plate to plate and for assays run on different days. Antibodies and assay reagents known in the art were used. Because of potential lot-to-lot variability of protein standards and antibodies, each lot of reagents used in the immunoassays may be standardized.

Of the 40 biomarkers, 33 were determined to be statistically different between NSCLC for all stages and high-risk control samples (P<0.05) using the Wilcoxon rank sum test. The 33 biomarkers are as follows: CXCL1 (GROα), CXCL3 (GROγ), CXCL5 (ENA-78), CCL1 (1309), CXCL9 (MIG), CXCL11 (I-TAC), CXCL12 (SDF-1), CCL3 (MIP-1α), CCL4 (MIP-1β), CCL5 (RANTES), CCL11 (eotaxin), CCL15 (MIP1δ), CCL19 (MIP3β), CCL22 (MDC), IL-2, IL-3, IL-4, IL-6, IL-7, IL-10, IL-12B (p40), IL-12 (p70), IL-13, IL-15, IL-17, GCSF, GM-CSF, INF-γ, IL-1α, IL-1β, IL1Ra, TNFβ, and VEGF.

Of the 40 biomarkers, 21 were determined to be statistically different between stage 1 NSCLC samples and high-risk control samples (p<0.05) using the Wilcoxon rank sum test. The 21 biomarkers are as follows: CXCL1 (GROα), CCL2 (MCP-1), CXCL9 (MIG), CCL3 (MIP-1α), CCL4 (MIP-1β), CCL5 (RANTES), CCL15 (MIP1δ), CCL22 (MDC), IL-2, IL-7, IL-10, IL-12B (p40), IL-12 p70, IL-13, IL-15, IL-17, GCSF, INF-γ, IL-10, IL1Ra, TNFβ, and VEGF.

Then two types of diagnostic models were constructed. The first type is a logistic regression model using small subsets of the markers. The second type combines the whole set (33) of significant markers (this was done for the all stages scenario).

For the first type, subsets of the markers were chosen for the two scenarios (all stages or stage I) using stepwise logistic regression. This resulted in the 4 marker model for all stages and the 3 marker model for stage I. In these logistic regression models the markers were entered into the model as continuous variables (that is there was no marker specific cut-points or categorizations). The logistic regression outputs a predicted probability of cancer for each subject based on a weighted combination of the markers in the model.

Specific details of logistic regression models: Logistic regression models the log odd (or logit). The odds defined as the ratio of P_{z}/(1−P_{z}) where P_{z} is the probability of cancer given the set of biomarkers. In a model with P number of predictors, the regression equation is: ln(odds)=α+β₁X₁+β₂X₂+ . . . +β_{P}X_{P}+ε
a. Where α is the intercept term in the model, the βi terms are the regression coefficient for the ith biomarker and the Xi is the value for the ith biomaker. The unknown parameters a and the βi (regression coefficients in the logistic regression model) are estimated by maximum likelihood using a method common to all generalized linear models as known in the art. The maximum likelihood estimates were computed numerically by using iteratively reweighted least squares. In this case, PROC LOGISTIC in the statistical software package SAS (SAS Institute Inc., Cary, N.C.) was to compute the estimates for the a and the βi that are given in the tables below. The same technique is employed to compute the estimate of the intercept (α) as for the biomarker coefficients (βi).
b. The predicted probability of cancer from the model would then be:

$${P}_{Z} = \frac{{e}^{α + {β}_{1} {X}_{1} + {β}_{2} {X}_{2} + … + {β}_{P} {X}_{P}}}{1 + {e}^{α + {β}_{1} {X}_{1} + {β}_{2} {X}_{2} + … + {β}_{P} {X}_{P}}}$$

c. Therefore, once the estimated regression coefficients are obtained, one can compute the sum of the products of the coefficients with their corresponding biomarker concentration values based on the formulation above to compute predicted probabilities.

The ROC curve was constructed for these two models by examining a number of cut-points of the predicted probabilities. The sensitivity and specificity indicated below is based on finding the cut-point of the predicted probability that maximizes the sum of the sensitivity plus specificity (e.g. maximizing Youden's J statistic).

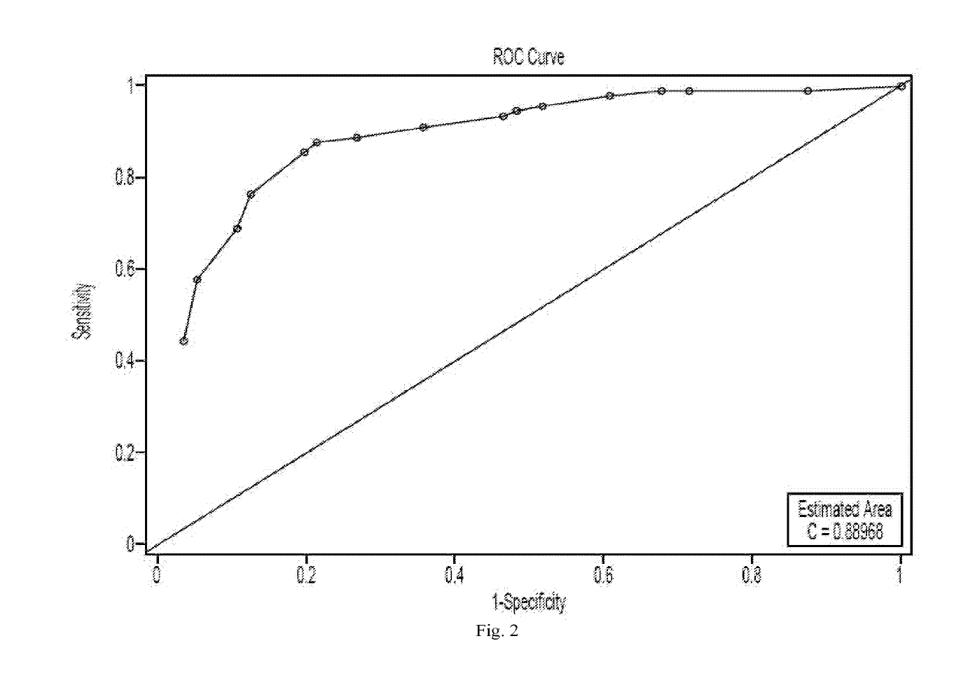

In particular, a panel consisting of only 4 biomarkers, i.e. VEGF, GCSF, MIG and RANTES, was used to create a predictive profile model of stages I-IV NSCLC vs. control (non-NSCLC). These biomarkers were combined together to compute predicted probability of cancer status based on logistic regression. For this case the releveant coefficients are provided in the table below. FIG. 2 is a ROC curve for the logistic regression model of stages I-IV NSCLC vs. control (non-NSCLC) using 4 biomarkers, i.e. VEGF, GCSF, MIG and RANTES. This model provides a sensitivity of 88%, a specificity of 79% and an AUC of 0.89.

| | Coefficients |
|---|---|
| Intercept | −5.20 |
| VEGF | 1.01 |
| GCSF | 1.40 |
| MIG | 2.30 |
| RANTES | 1.85 |

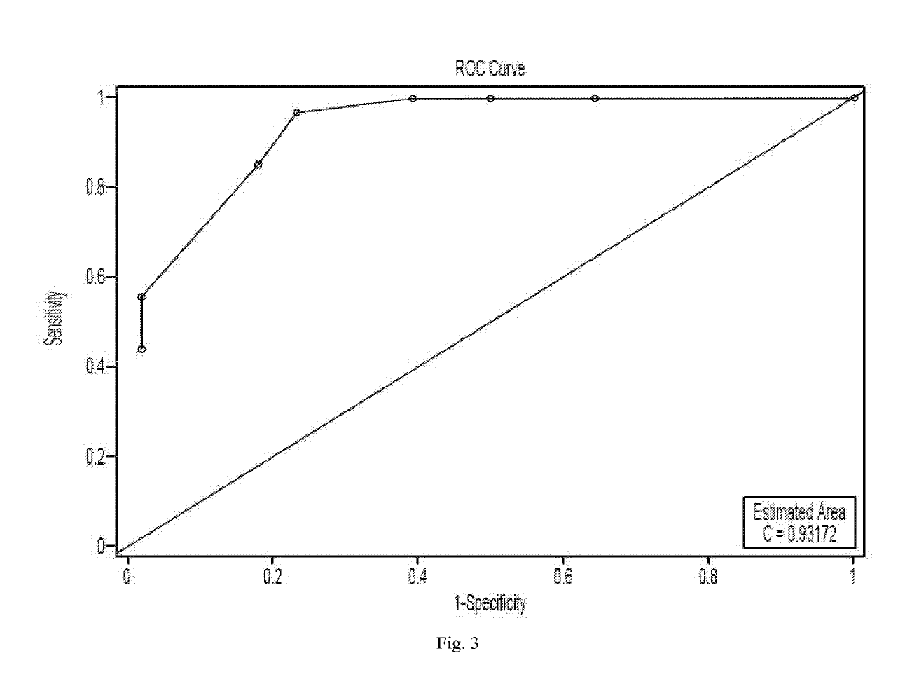

The concentrations of IL-2, IL-3 and MDC in serum samples of stage I NSCLC subjects and high-risk control subjects were used to construct a logistic regression model of stage I NSCLC vs. control (non-NSCLC). FIG. 3 is a ROC curve for a predictive profile model of stage I NSCLC vs. control (non-NSCLC) using 3 biomarkers, i.e. IL-2, IL-3 and MDC. This model provides a sensitivity of 97%, a specificity of 77%, and an AUC of 0.93.

| | Coefficients |
|---|---|
| Intercept | −3.41 |
| IL-2 | 2.76 |
| IL-3 | 2.53 |
| MDC | 1.87 |

For the second type, which is a simple voting model, each biomarker was categorized into high or low categories. This categorization was based on a biomarker specific cut-point which was the median value for that marker across the whole subject pool (NSCLC and controls). A summary score was then created by adding up the number of markers that were greater than their cut-point. This summary score was then used to create an ROC curve and the sensitivity and specificity for the summary score was assessed by identifying the value of the summary score which resulted in the maximum of the sum of the sensitivity and specificity.

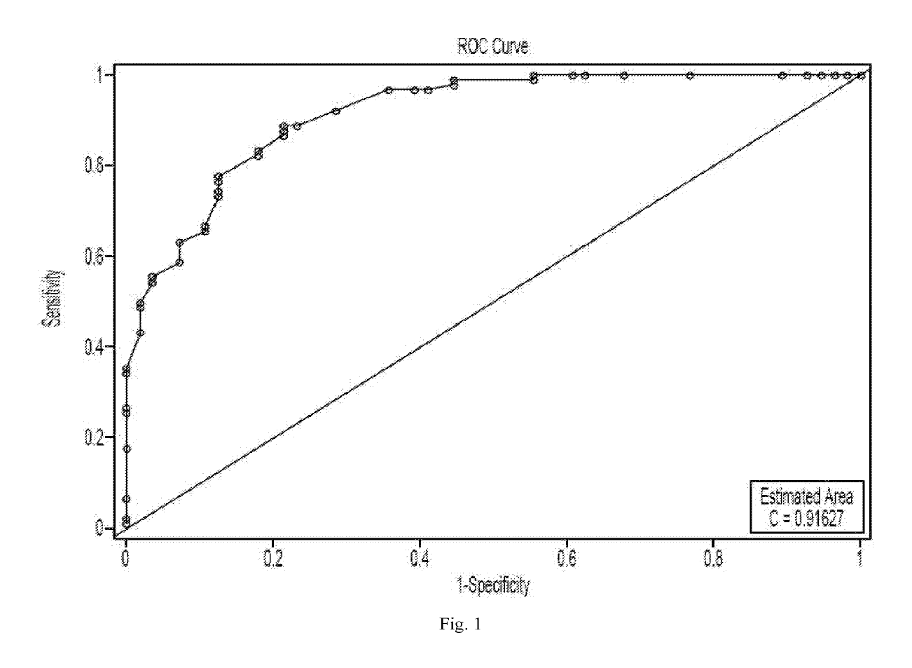

In particular, in order to provide a predictive model for the presence of NSCLC, each biomarker concentration was categorized as high or low based on a threshold computed for the given biomarker. This threshold was established based on the median of each biomarker across the combined subject set of NSCLC and high-risk controls. Next, an overall marker score, which is the number of biomarkers higher than the median value for each specific marker, was computed for each sample. This median of each marker was the median value for the marker across the entire cohort (including the overall marker score input into a logistic regression model for computing an individual subject's cancer risk probability). Then the sensitivity, specificity and area under the ROC curve (AUC) of given panels of selected biomarkers were calculated using the cut-point that maximized Youden's J statistic (i.e. the sum of the sensitivity+specificity) for the biomarker scores over all of the 33 significant biomarkers from the NSCLC all stages vs control. Based on the cut-off for the overall marker score a sensitivity of 87% and a specificity of 78% were obtained for all stages of lung cancer detection. Additionally, the AUC for this risk predictor is 0.92. The area under the ROC curve provides a single index that summarizes the diagnostic ability of the marker under consideration. The area under the curve is computed by performing numerical integration of the ROC curve. The computations were performed using the SAS statistical software package (SAS Institute Inc., Cary, N.C.). FIG. 1 shows a ROC curve for this predictive model for NSCLC vs. control (non-NSCLC).

| | Coefficients |
|---|---|
| Intercept | −5.43 |
| Overall Marker Score | 0.32 |

Thus, for a given set of biomarkers, once their regression coefficients and the intercept term are obtained, the probability of lung cancer may be calculated using the biomarker concentration values obtained from a sample. For example, amounts of VEGF, GCSF, MIG and RANTES in a blood, plasma, or serum sample from a subject at high risk for lung cancer are determined and the biomarker concentration values are calculated. Then the regression coefficients and the intercept value for these 4 biomarkers are used to calculate the predicted probability of lung cancer. For example, the regression coefficients and the intercept value provided above are used along with the biomarker concentration values to obtain the predicted probability, Pz, above. A Pz value near 0 or 0 indicates that the subject does not likely have lung cancer. A Pz value near 1 or 1 indicates that the subject likely has lung cancer. For example, a Pz value of 0.9 indicates that the subject has a 90% likelihood of having lung cancer.

Similarly, where the predictive model is for determining the probability of stage I NSCLC, e.g. using the model employing IL-2, IL-3 and MDC, the amounts of IL-2, IL-3 and MDC in a blood, plasma, or serum sample from a subject at high risk for lung cancer are determined and the biomarker concentration values are calculated. Then the regression coefficients and the intercept value for the given biomarkers are used to calculate the predicted probability of stage I NSCLC. A Pz value near 0 or 0 indicates that the subject does not likely have stage I NSCLC. A Pz value near 1 or 1 indicates that the subject likely has stage I NSCLC. For example, a Pz value of 0.2 indicates that the subject has a 20% likelihood of having stage I NSCLC.

Analysis of clinical specimens from stage I NSCLC subjects and high-risk control subjects revealed increased expression of pro-angiogenic and pro-inflammatory cytokines in the NSCLC subjects compared to high-risk control subjects and diminished expression of anti-angiogenic and anti-inflammatory cytokines in the NSCLC subjects. Based on these results, one or more additional protein biomarkers associated with anti-angiogenic and anti-inflammatory biochemical pathways, such as those set forth in Table 2 may be included in methods and devices according to the instant invention.

TABLE 2

| Name | Complete name and reference citation |
|---|---|
| Amphiregulin | Shoyab et al. (1989) Science 243 (4894 Pt 1): 1074-6. |
| Lipocalin | Flower et al. (1993) Protein Sci. 2 (5): 753-761. |
| LIF | Leukemia inhibitory factor, Patterson (1994) PNAS |
| | USA 91 (17): 7833-5. |
| sE-cadherin | Soluble E-Cadherin, Katayama M., (1994) Br. J. Cancer |
| | 69(3): 580-5 |
| CXCL7 | Chemokine (C—X—C motif) ligand 7, Schenk (2002) |
| (CTAP III) | Journal of Immunology, 169: 2602-2610 |
| SCF | Stem cell factor Geissler (1991) Somat Cell Mol Genet. |
| | Mar; 17(2): 207-14 |
| TGF-β | Transforming growth factor beta, Coffey RJ (1986) |
| | Cancer Research 46(3): 1164-9 |
| PDGF-BB | Platelet-derived growth factor subunit B, Ratner et al. |
| | (1985) Nucleic Acids Res 13 (14): 5007-18. |
| TRAIL | TNF-related apoptosis-inducing ligand, Wiley et al. |
| | (1995) Immunity 3 (6): 673-82. |
| MMP-9 | Matrix metallopeptidase 9, Nagase et al. (1999) J. Biol. |
| | Chem. 274 (31): 21491-4. |
| MIF | Macrophage migration inhibitory factor, Weiser (1989) |
| | PNAS USA 86 (19): 7522-6. |

The sequences of the above-referenced proteins are herein incorporated by reference in their entirety.

Therefore, the methods of the present invention may be used to determine whether a high-risk subject should be subjected to further diagnostic procedures to detect lung cancer. For example, where the biomarker expression profile obtained from a subject is the same or substantially similar to a biomarker expression profile that is indicative of lung cancer, one may determine that the subject should undergo further diagnostic testing such as an imaging study, fiberoptic bronchoscopy, cytologic examination of materials obtained via endobronchial brushings, bronchoalveolar lavage and endo- and transbronchial biopsies, or a combination thereof.

The methods of the present invention may also be used to monitor lung cancer treatments and/or cancer progression/remission. For example, a biomarker expression profile that is the same or substantially similar to a biomarker expression profile that is indicative of a high risk subject that does not have lung cancer (i.e. the biomarker expression profile changes from being the same or substantially similar to a biomarker expression profile that is indicative of lung cancer) could be used to indicate that the given treatment was successful and/or remission. The subject can then be treated based on the amounts of the biomarkers. For example, if the biomarker expression profile is indicative of lung cancer, the subject can them be subjected to one or more cancer treatments known in the art.

The methods of the present invention may be used to diagnose lung cancer or monitor a subject for lung cancer who exhibits an indeterminate pulmonary nodule. For example, where a subject exhibits an indeterminate pulmonary nodule, but has a biomarker expression profile that is the same or substantially similar to a biomarker expression profile that is indicative of lung cancer, be subject may be categorized as having lung cancer, closely monitored for developing lung cancer, and or subjected to further diagnostic tests for lung cancer.

In addition to assaying protein biomarkers, the expression levels of various microRNAs (miRNAs) in serum and/or plasma samples from lung cancer subjects and high-risk control subjects were measured. Specifically, the expression levels of a let-7f, miR-16, miR-17, miR-21, miR-24, miR-25, miR-34a, miR-106a, miR-125a-3p, miR-126*, miR-128, miR-146b-5p, miR-155, miR-199a, miR-200c, miR-221 and miR-222 were assayed in a subset of the serum samples that were used in the protein biomarker assays described above. The accession numbers of each of the miRNAs are set forth in Table 3 as follows:

TABLE 3

| Name | Accession Number |
|---|---|
| let-7f | MIMAT0000067 |
| miR-16 | MIMAT0000069 |
| miR-17 | MIMAT0000070 |
| miR-21 | MIMAT0000076 |
| miR-24 | MIMAT0000080 |
| miR-25 | MIMAT0000081 |
| miR-34a | MIMAT0000255 |
| miR-106a | MIMAT0000103 |
| miR-125a-3p | MIMAT0004602 |
| miR-126* | MIMAT0000444 |
| miR-128 | MIMAT0000424 |
| miR-146b-5p | MIMAT0002809 |
| miR-155 | MIMAT0000646 |
| miR-199a-3p | MIMAT0000232 |
| miR-200c | MIMAT0000617 |
| miR-221 | MIMAT0000278 |
| miR-222 | MIMAT0000279 |

The sequences of the above-referenced miRNAs as set forth in the miRBase database, Release 16 (Sept 2010) which is hosted and maintained in the Faculty of Life Sciences at the University of Manchester with funding from the BBSRC, and was previously hosted and supported by the Wellcome Trust Sanger Institute are herein incorporated by reference in their entirety.
See miRBase: tools for microRNA genomics, Griffiths-Jones et al. NAR 2008 36(Database Issue): D154-D158; miRBase: microRNA sequences, targets and gene nomenclature. Griffiths-Jones et al. NAR 2006 34(Database Issue): D140-D144; and The microRNA Registry, Griffiths-Jones NAR 2004 32 (Database Issue): D109-D111, which are herein incorporated by reference in their entirety.
The miRBase database is available at WorldWideWeb(dot)mirbase(dot)org where “WorldWideWeb” = “www” and “(dot)” = “.”

It was found that miR-21, miR-25, miR-34a and miR-200c were significantly differentially expressed between stage 1 NSCLC subjects and high-risk controls (p<0.05) and miR-146b gave a p value of <0.08. Thus, the methods and devices of the present invention employing some or all of the protein biomarkers as disclosed herein may be multiplexed with microRNA (miRNA) assays. For example, the concentrations of a given set of protein biomarkers and the concentrations of a given set of miRNAs may be measured in a test serum and/or plasma sample of a subject and then the subject is diagnosed as having lung cancer based on the concentrations of the protein biomarkers and the miRNAs. In some embodiments, one or more miRNAs selected from the group consisting of miR-21, miR-25, miR-34a, miR-200c and miR-146b are assayed. In some embodiments, about 4-8 protein biomarkers and one or more of the miRNAs as described herein may be used to detect or diagnose the presence or absence of lung cancer in a subject. For example, the concentrations of CXCL3, CCL3, CCL15, IL-6, GMCSF, IL1α, IL1β, VEGF, miR-21, miR-25, miR-34a, and miR-200c in a serum sample of a subject may be used to detect or diagnose the presence or absence of lung cancer, such as stage 1 NSCLC, in the subject.

In embodiments which include miRNA assays, the miRNA expression levels may be assayed using methods known in the art. For example, the following protocol can be used. RNA is be isolated from 200 μl of human serum using miRNEASY kit (Qiagen, Valencia, Calif.) according to the modified manufacturer's protocol for the liquid samples. 200 μl of serum is thawed on ice and mixed thoroughly by vortexing with 5 volumes of QIAZOL LYSIS REAGENT from the MIRNEASY miRNA isolation kit and is subsequently incubated at room temperature for 5 minutes. At this point, synthetic C. elegans miRNAs cel-miR-39, cel-miR-54 and cel-miR-238 (synthesized by IDT, Coralville, Iowa) is added to the samples as a mixture of 25 fmol of each miRNA in a 5 μl total volume using methods known in the art to serve as normalization controls. One volume (200 μl) of chloroform is then added to each sample. The resulting suspensions are vortexed for 15 seconds and spun for 15 minutes at 12000 g at 4° C. The aqueous phase is collected, mixed with 1.5 volume of 100% ethanol and passed through a column provided with the kit. The column is washed and RNA is eluted with 40 μl of elution buffer according to the manufacturer's protocol. miRNA expression is determined by quantitative RT-PCR using Qiagen's MISCRIPT platform. Briefly, 10 μl of total RNA eluted from the MIRNAEASY column is polyadenylated in vitro and reversely transcribed utilizing MISCRIPT REVERSE TRANSRIPTION KIT. qPCR is performed using QUANTITECT SYBR GREEN mix and primers as recommended by the manufacturer. PCR reactions and data analysis is performed using ICYCLER and IQ5 software package (Bio-Rad, Hercules, Calif.) respectively. Data is normalized to the spike-in synthetic miRNA controls. All sample groups in the PCR experiments are run in triplicate and randomized to prevent experimental bias.

The methods and devices of the present invention employing some or all of the protein biomarkers, with or without one or more miRNAs, as disclosed herein may also be multiplexed with other diagnostic methods known in the art for detecting or diagnosing NSCLC and/or other cancers, such as imaging studies, fiberoptic bronchoscopies, cytologic examinations, bronchoalveolar lavage and endo- and transbronchial biopsies, transthoracic biopsies, exploratory thoracotomies, and the like.

Although the experiments described herein were performed on plasma and serum samples, the methods and devices of the present invention may be performed using whole blood samples. In addition, although the experiments described herein were performed using a specific high risk control group, i.e. former smokers at risk for lung cancer (≧30 pack years, age ≧45, smoking cessation of at least 1 year), the methods and devices described herein may be applied to other high risk subjects, e.g. current smokers, younger subjects, subjects who smoke or smoked less than 30, e.g. 20-29, packs per year, ceased smoking less than one year prior to being tested, or a combination thereof.

Devices according to the present invention comprise one or more substrates having capture reagents immobilized thereon, e.g. antibodies which specifically bind a given set of protein biomarkers and/or miRNAs and/or nucleic acid molecules which hybridize to a given set of miRNAs. After the substrate is contacted with a sample, the amount of each protein biomarker and/or miRNA captured by the capture reagent may be determined using methods known in the art.

Kits according to the present invention comprise reagents for assaying the amounts of at least three of the protein biomarkers as disclosed herein, e.g. at least three of the following protein biomarkers: VEGF, CGSF, MIG, RANTES, IL-2, IL-3 and MDC, packaged together. The kits may further comprise tools and devices for collecting and storing samples obtained from subjects.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of diagnosing the likelihood of a subject as having a lung cancer which comprises
measuring the amounts of at least three of the following protein biomarkers: VEGF, CGSF, MIG, RANTES, IL-2, IL-3 and MDC, in a blood, serum or plasma sample obtained from the subject using capture reagents which specifically bind the biomarkers,
determining whether the amounts measured are indicative of the presence or absence of the lung cancer or the subject as being at high risk for the lung cancer using the following logistic regression model
$${P}_{Z} = \frac{{e}^{α + {β}_{1} {X}_{1} + {β}_{2} {X}_{2} + … + {β}_{P} {X}_{P}}}{1 + {e}^{α + {β}_{1} {X}_{1} + {β}_{2} {X}_{2} + … + {β}_{P} {X}_{P}}}$$
where P_{z} is a predicted probability, P is the number of biomarkers, α is the intercept term, βi terms are a regression coefficient for the ith biomarker, and Xi terms are the value for the ith biomarker; and
diagnosing the subject as (1) not likely having the lung cancer where the predicted probability is near 0 or 0, (2) likely having the lung cancer where the predicted probability is near 1 or 1, or (3) having a N % likelihood of having the lung cancer where the predicted probability is n and 0<n>1 and N=n×100.

2. (canceled)

3. The method of claim 1, wherein the lung cancer is non-small cell lung cancer.

4. The method of claim 3, wherein the amounts of VEGF, GCSF, MIG and RANTES are measured and used in the logistic regression model to calculate the predicted probability.

5. The method of claim 1, wherein the lung cancer is stage I non-small cell lung cancer.

6. The method of claim 5, wherein the amounts of IL-2, IL-3 and MDC are measured and used in the logistic regression model to calculate the predicted probability.

7. The method according to claim 1, wherein the subject is categorized as being at high risk for lung cancer.

8. The method according to claim 1, wherein the subject smokes or has smoked at least 20 packs of cigarettes, preferably at least 30 packs of cigarettes per year and is at least 35 years of age, preferably at least 45 years of age.

9. The method according to claim 1, wherein the subject is diagnosed as likely having the lung cancer where the predicted probability is greater than or equal to 0.6, preferably greater than or equal to 0.7, more preferably greater than or equal to 0.8, most preferably greater than or equal to 0.9.

10. The method according to claim 1, wherein the subject is diagnosed as not likely having the lung cancer where the predicted probability is less than or equal to 0.4, preferably less than or equal to 0.3, more preferably less than or equal to 0.2, most preferably less than or equal to 0.1.

11. The method according to claim 1, which further comprises
determining the amounts of one or more of the following protein biomarkers: CXCL1 (GROα), CXCL3 (GROγ), CXCL5 (ENA-78), CCL1 (1309), CXCL11 (I-TAC), CXCL12 (SDF-1), CCL3 (MIP-1α), CCL4 (MIP-1β), CCL11 (eotaxin), CCL15 (MIP16), CCL19 (MIP3β), IL-4, IL-6, IL-7, IL-10, IL-12B (p40), IL-12 (p70), IL-13, IL-15, IL-17, GM-CSF, INF-γ, IL-1α, IL-1β, IL1Ra, TNFβ, Lipocalin, LIF, sE-cadherin, CXCL7 (CTAP III), SCF, TGF-β, PDGF-BB, TRAIL, MMP-9, and MIF and determining whether the amounts are indicative of the lung cancer.

12. The method according to claim 1, which further comprises
determining the amounts of one or more of the following protein biomarkers: CXCL3 (GROγ), CCL3 (MIP-1α), CCL15 (MIP1δ), IL-6, IL-1α, and IL-1β, and
determining whether the amounts are indicative of the lung cancer.

13. The method according to claim 1, which further comprises
determining the amounts of one or more miRNAs selected from the group consisting of miR-21, miR-25, miR-34a, miR-200c and miR-146b, and
determining whether the amounts are indicative of the lung cancer.

14. A method of monitoring or treating a subject who is at high risk of having a lung cancer, who has the lung cancer or who has had the lung cancer, which comprises
diagnosing the subject in accordance with claim 1, and then subjecting the subject to further diagnostic procedures to detect the lung cancer and/or subjecting the subject to a cancer treatment where the subject is diagnosed as likely having the lung cancer.

15. A device which comprises at least three capture reagents immobilized on one or more substrates, which each capture reagent specifically binds one protein biomarker selected from the group consisting of: VEGF, CGSF, MIG, RANTES, IL-2, IL-3 and MDC.

16. A kit which comprises reagents for assaying the amounts of at least three of the protein biomarkers as disclosed herein, e.g. at least three of the following protein biomarkers: VEGF, CGSF, MIG, RANTES, IL-2, IL-3 and MDC, packaged together.

17. (canceled)
